(19) United States
(12) Patent Application Publication
(10) Pub. No.: US 2022/0354441 A1
(43) Pub. Date: Nov. 10, 2022

(54) SYSTEMS FOR MANAGING ALARMS FROM MEDICAL DEVICES

(71) Applicant: GE Precision Healthcare LLC, Wauwatosa, WI (US)

(72) Inventors: Nickolas MacDougall, Wauwatosa, WI (US); Aaron D. Hart, Cedarburg, WI (US); Tabatha Shuster, Chattanooga, TN (US)

(21) Appl. No.: 17/729,535

(22) Filed: Apr. 26, 2022

Related U.S. Application Data
(60) Provisional application No. 63/183,895, filed on May 4, 2021.

(51) Int. Cl. A61B 5/00

(52) U.S. Cl. CPC A61B 5/7475; A61B 5/7264; A61B 5/7425; A61B 5/743; A61B 5/746

(57) ABSTRACT

A system for managing an alarm based on active data from a medical device and based on supplemental data includes: a display configured to present select active data from among a plurality of active datasets comprising the active data and present select supplemental data from among a plurality of supplemental datasets comprising the supplemental data; and a processing system configured to: trigger an alarm based on the active data, determine which of the plurality of supplemental datasets to present as the select supplemental data in the second window based on the alarm triggered; control the display to display the select supplemental datasets in the second window; and based on receiving a user alarm input indicating that the alarm is false, provide an interface for updating the one or more alarm limits based on the displayed select supplemental data to manage the alarm of the medical device.

### PRIORITY

This application is based on and claims priority to U.S. Provisional Patent Application No. 63/183,895 filed on May 4, 2021 at the United States Patent and Trademark Office, the disclosure of which is hereby incorporated by reference.

### FIELD

The present disclosure relates to monitoring medical patients, and in particular to managing alarms from medical devices providing data corresponding to the patients.

### BACKGROUND

Alarm fatigue causes barriers to necessary treatment in the acute care setting. Alarms may be triggered on medical devices for a wide variety of causes, some involving medical parameters such as heart rate, CO2 level, or blood pressure, for example, and others being technical parameters such as no or poor incoming signal, low battery, and/or the like. While the alarms are intended to bring the users attention to a potential or actual issue with the patient or medical device itself, may times the alarms are merely nuisances and do not require clinical intervention. For example, various alarms may go off when a patient is temporarily disconnected from a medical device, such as replacing hoses on a ventilator or disconnecting the patient from a monitoring device to use the restroom. Due to the sheer volume of alarms present for a medical device over time, it is difficult for a caregiver to quickly recognize when a true condition requiring clinical intervention is present amongst the noise of all other conditions, including false alarms, and thus there is a risk to the patient that these true alarm conditions are not recognized and acted upon quickly.

### SUMMARY

According to an aspect of the disclosure, a system for managing an alarm based on active data from a medical device and based on supplemental data, wherein the active data has one or more alarm limits for triggering the alarm, may include: a display having a first window and a second window, wherein the first window is configured to present select active data from among a plurality of active datasets comprising the active data, the second window being configured to present select supplemental data from among a plurality of supplemental datasets comprising the supplemental data, the alarm being triggered based on the active data; a processing system configured to: determine which of the plurality of supplemental datasets to present as the select supplemental data in the second window based on the alarm triggered; control the display to display the select supplemental datasets in the second window; and based on receiving a user alarm input indicating that the alarm is false, provide an interface for updating the one or more alarm limits based on the displayed select supplemental data to manage the alarm of the medical device.

The select active data may include a heart rate, and wherein the plurality of supplemental data includes at least one of an A/V feed of a room, laboratory results, medication ordered, and medication administered.

The plurality of supplemental data may include at least one of medication ordered and medications administered. The system may further include a medication information database accessible by the processing system, wherein the processing system analyzes the alarm in view of the at least one of the medication ordered and the medication administered and the medication information database to determine whether to present the at least one of the medication ordered and the medication administered among the select supplemental data in the second window.

The processing system may be further configured to, based on receiving a user alarm input indicating that the alarm is false, change which of the plurality of supplemental datasets to present among the select supplemental data in the second window based on the alarm being indicated to be false.

The processing system may be further configured to present a prompt on the interface requesting the user alarm input based on the alarm being present on the medical device.

The processing system may be further configured to determine, based on a trained neural network model, which of the plurality of active datasets to be present as the select active data based on the alarm of the medical device, the neural network model being trained based on a correlation between false positive alarms and supplemental datasets.

The medical device may be among a plurality of medical devices corresponding to a plurality of patients, wherein the active data and the supplemental data for each of the plurality of patients are receivable and presentable on the interface, and wherein the processing system is further configured to assign and present a priority rating to each of the plurality of patients based on the alarm present on the medical device corresponding thereto.

The processing system may be further configured to receive a patient selection selecting among the plurality of patients, and wherein the select supplemental data presented on the interface corresponds to which of the plurality of patients is selected.

The processing system may be configured to control the display to display selected historic data in a third window, wherein the selected historic data is among a plurality of historic datasets received from the medical device over time, wherein the processing system is further configured to: determine which of the plurality of historic datasets to present as the selected historic data based on the alarm present for the medical device, control the display to display the selected historic data in the third window; and based on displaying the selected historic data, provide the interface for updating the one or more alarm limits based on the selected historic data to manage the alarm of the medical device.

The processing system may determine which of the plurality of supplemental datasets to present as the select supplemental data based also on the selected historic data presented.

The processing system may be further configured to, based on receiving the user alarm input indicating that the alarm is false: identify patterns in the historic data by comparing the indication that the alarm is false to the selected historic data, and store the identified patterns as false alarm predictions that are accessible by the processing system.

The processing system may be further configured to: analyze the historic data using the false alarm predictions; and based on the patterns being identified in the historic data, present a suggestion to check whether the alarm is false.

The processing system may be further configured to identify patterns within the selected historic data and to correspondingly present medication administered among the select supplemental data in the second window.

The processing system may be further configured to control the display to display alarm frequency data in a fourth window and determine the alarm frequency data based on how often the alarm is present within the selected historic data presented in the third window.

The alarm frequency data may be further presented as a function of how much the selected historic data was outside the one or more alarm limits corresponding to the alarm. The one or more alarm limits include an upper limit and a lower limit, and wherein the alarm frequency data is further presented as a function of how often the selected historic data was above the upper limit and below the lower limit.

The alarm frequency data may further present the upper limit and the lower limit, and the processing system may be further configured to receive proposed change inputs for the upper limit and the lower limit, and wherein the alarm frequency data is updateable based on the user alarm inputs received.

A plurality of possible actions may be accessible by the processing system, and the processing system may be further configured to control the display to display a fifth window that presents select actions on the interface, wherein the select actions are among the plurality of possible actions and are selected by the processing system based on the alarm, the select actions comprising sending a communication to a caregiver presenting the alarm and requesting confirmation of the one or more alarm limits corresponding thereto.

The processing system may be further configured to control the display to display a third window that presents selected historic data on the interface, wherein the selected historic data is among a plurality of historic datasets received from the medical device, wherein the processing system determines which of the plurality of historic datasets to present among the selected historic data based on the alarm on the medical device, and wherein the select actions include presenting suggested adjustments to the one or more alarm limits based on the selected historic data.

According to another aspect of the disclosure, a patient monitoring device, may include: a communication interface configured to receive and send data; a memory storing one or more instructions; a display for displaying information to a user; a user interface configured to receive inputs from the user; and a processor configured to execute the one or more instructions to: receive active data from one or more medical devices through the communication interface; trigger an alarm based on the active data exceeding a threshold; based on the alarm being triggered, select, using a selection model, one or more types of supplemental data, from among a plurality of supplemental data types based on a type of the triggered alarm; and automatically control the user interface to display the selected one or more types of supplemental data, the selected one or more types of supplemental data being displayed in conjunction with the active data.

According to another aspect of the disclosure, a method of monitoring medical devices may include: receiving active data from a plurality of medical devices; displaying the active data on a display; triggering an alarm based on the active data from a medical device, among the plurality of medical devices, reaching one or more alarm limits; based on the alarm being triggered, providing an interface for selecting whether the alarm is true of false; and based on receiving a user alarm input indicating that the alarm is false: selecting, using a selection model, one or more types of supplemental data, from among a plurality of supplemental data types based on a type of the triggered alarm; and providing an interface for updating the one or more alarm limits to manage the alarm of the medical device.

### BRIEF DESCRIPTION OF THE DRAWINGS

The present disclosure is described with reference to the following drawings.

FIG. 1 is an overhead view of a system for managing alarms while overseeing the care of multiple patients in an exemplary setting, according to an example;

FIG. 2 depicts an exemplary interface within a system for managing alarms according to an example;

FIG. 3 is a process flow diagram for managing alarms according to an example;

FIG. 4 is an example of a process flow for managing alarms according to an example;

FIG. 5 is an example of a process flow for managing alarms, according to an example;

FIG. 6 is a specific example of a process flow for managing alarms, this time for a situation in which multiple alarms are triggered simultaneously, according to an example; and

FIG. 7 is an exemplary schematic for a control system such as may be used to manage alarms, according to an example.

### DETAILED DISCLOSURE

The present inventors have recognized issues with systems and methods presently known in the art with respect to preventing and managing alarm fatigue for medical devices. In particular, the present inventors have recognized that reducing false alarms (in other words, increasing the likelihood of an alarm being a “true” alarm) reduces alarm fatigue and enhances a caregiver's ability to quickly identify and address those medical conditions requiring clinical intervention. Systems and methods employed today set alarm limits and manage alarms through analysis of only the parameter causing the alarm. In other words, blood pressure limits are set to pre-established limits, and/or adjusted automatically or through caregiver modification, based on consideration of that patient's particular blood pressure readings. For example, a caregiver may respond to a high blood pressure alarm, look at the current blood pressure waveforms, and conclude that not intervention is required. The caregiver will then either “silence” the alarm (a temporary answer that does not reduce the incidences of future, false alarms), or in some cases modify an alarm limit such that the current, acceptable reading no longer triggers the alarm.

It should be recognized that medical devices include many different types of devices, including but not limited to ventilators, IV pumps, and monitoring devices.

The systems and methods presently disclosed provide for prioritizing alarms and reducing the number of false alarms through the additional use of supplemental or collateral data, rather than simply assessing alarms and setting alarm limits based on the alarming data itself. For example, the present inventors have recognized that optimally setting the alarm limits for a heart rate is not limited to information related to that heart rate itself, but may also be best informed through simultaneous display of other, supplemental data that may directly or indirectly impacts the heart rate. By identifying and prompting the caregiver to assess this supplemental data in addition to the alarming data (also referred to herein as the “active” data), alarm limits may be set to avoid the incidence of false alarms. Reducing the incidence of false alarms, and thus the incidences of alarms in general, the caregiver is better able to quickly identify and remediate conditions associated with true alarms that are no longer buried among the false alarms.

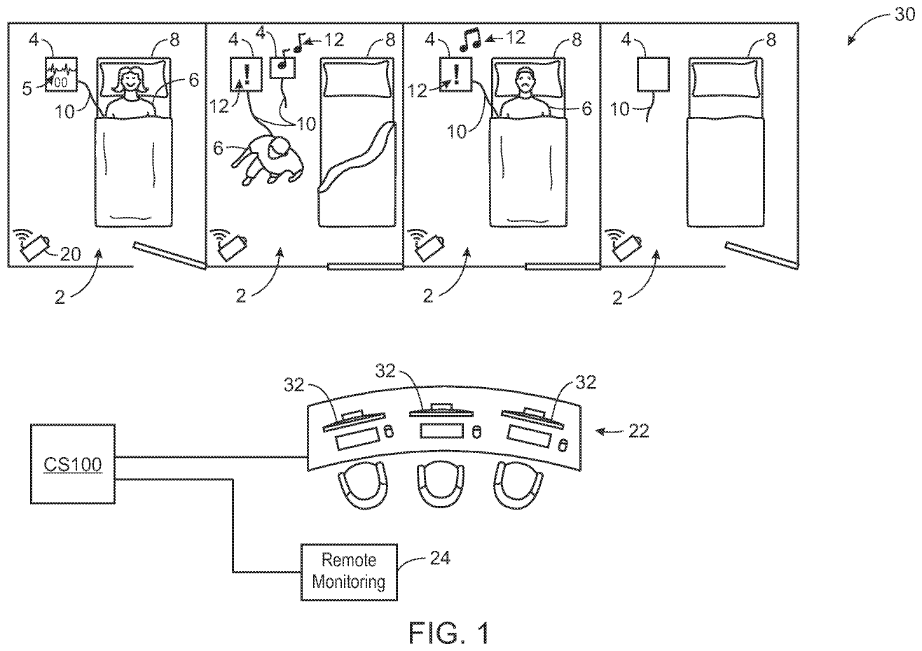

FIG. 1 depicts an example of a system for managing alarms, shown here in the context of a exemplary setting in which multiple patients are monitored by a central monitoring unit 22 and/or remote monitoring unit 24. In this example, four rooms 2 are shown having one or more medical devices 4 and beds 8. A camera 20 is provided in each of the rooms 2 for providing an audio/video (A/V) feed of the room 2, enabling a caregiver to watch/listen to the patient 6 without being physically in the room 2. The patient's 6 are connected to the medical devices via connections 10, which may be tubes for ventilation, leads for pulse oximeters, ECGs, EMGs, and EEGs, and/or the like in a manner presently known. The medical devices 4 have interfaces 5, which may include a display, buttons, keyboards, and/or speakers such as presently known. The two central rooms 2 are presently shown with alarms 12 being presented by the medical devices 4, which may be visual and/or auditory in nature. The presence of the alarm 12, as well as data from the medical device 4, camera 20, and/or other sources are provided for the central monitoring unit 22 and/or remote monitoring unit 24, for example though a control system CS100, which is discussed further below. According to an example, the central monitoring unit 22 and the control system CS100 may be a single device with component provided at separate locations.

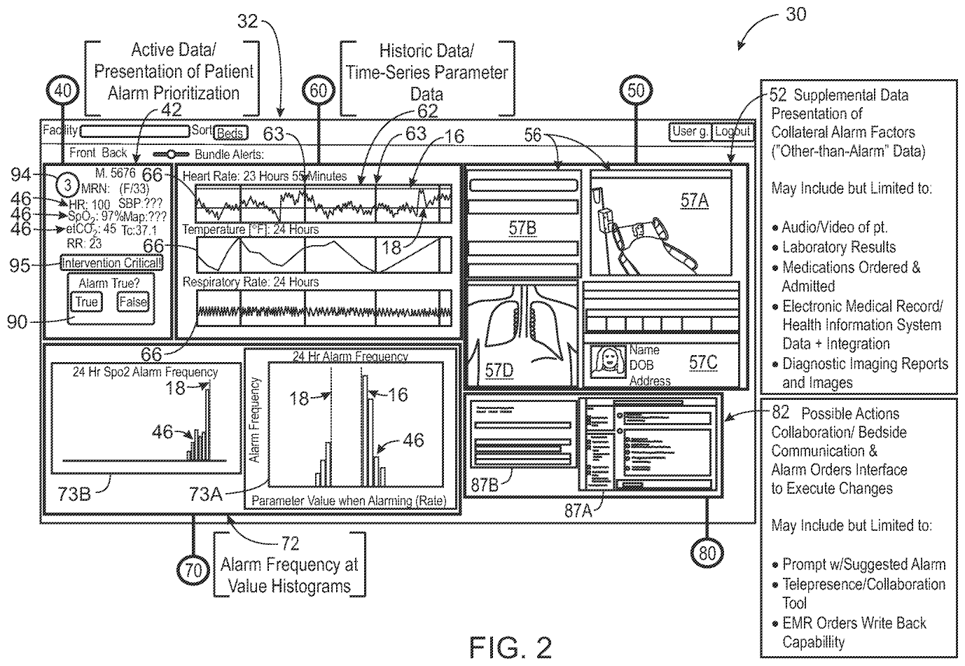

FIG. 2 depicts an exemplary interface 32 within a system 30 according to an example. As will become apparent, the system 30 provides for managing an alarm based on active data from the medical device, and also based on supplemental data. The active data has one or more alarm limits for triggering the alarm, whereby properly setting or adjusting the one or more alarm limits minimizes the incidences of false alarms, whereby reducing alarm fatigue. The system 30 includes an interface 32 that incorporates a number of modules therein.

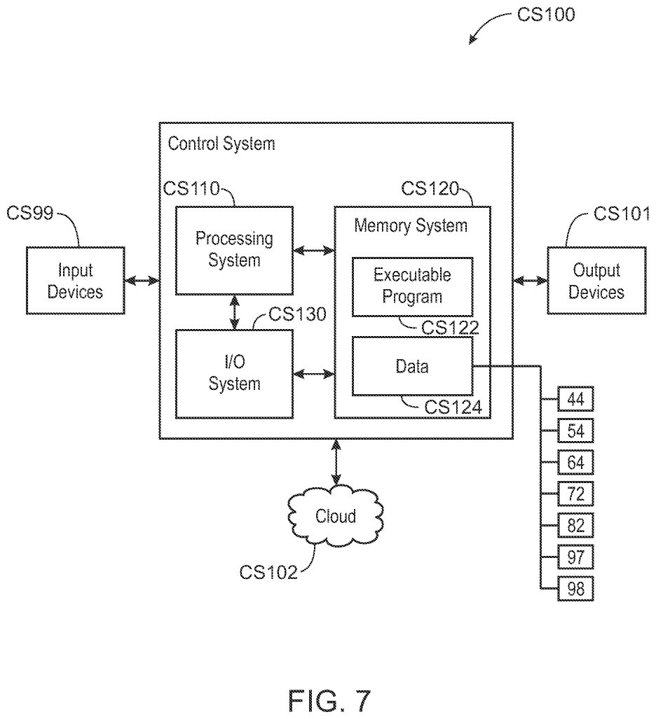

A first module 40 (or window) is configured to present active data 42, and particularly to present select active data 46 from within a plurality of active data sets 44 stored in a memory system CS120 (see FIG. 7). The active data 42 is provided by one or more medical devices 4 and is subject to one or more alarm limits, whereby the active data 42 being outside the alarm limits results in the alarm for the medical device 4, and particularly improperly set alarm limits resulting in false alarms. Examples of active data include heart rate, SpO2, etCO2, a respiratory rate, or other medical parameters typically generated and communicated from a medical device 4.

A second module 50 on the interface 32 is configured to present supplemental data 52, and particularly select supplemental data 56 from among a plurality of supplemental datasets 54 stored in the memory system CS120 (FIG. 7). The supplemental data 52 is data other than the active data 42, or in other words is data other than what is triggering the present alarm from the medical device 4.

The supplemental data 52 may come from the same medical device 4 as the active data 42 generating the alarm, and/or may come from other medical devices 4, or other sources altogether. Examples of supplemental data 52 include an A/V feed 57A taken from the camera 20 in the room 2 (FIG. 1), which allows the caregiver to view the patient 6 without being present in room 2. Additional examples of supplemental data 52 include information relating to medication ordered or medication administered 57B to the patient, caregiver notes 57C, imaging results 57D, information from an EMR/HIS data 57E (e.g., current diagnosis and/or underlying conditions), and/or lab results 57F. It will become apparent that this supplemental data 52 is not fixed, but identified by the system 30 responsive to the specific alarm or alarms going for a given patient. That is, a subset of available supplemental data options may be selected and displayed based on the alarm.

The selection determination may be made by the computing system CS100 or another processor in communication with the system. A prestored model may be used to make the selection determination. According to an example, the model may be a trained a neural network model. The model may be continuously trained based on the information provided by the system. For example, the model may be designed to learn that supplemental data types that result in a high rate of false positives are more valuable to a caregiver. As such, the model will learn to provide these supplemental data types having a high false positive rate for specific alarms by learning from previous information provided by the system, such selections of a caregiver. According to an example, the neural network model may be trained based on a correlation between false positive alarms and supplemental datasets.

The selection determination model may also include rules set by a clinician. For example, preset rules may correspond specific selection of supplemental data with specific alarms.

By way of non-limiting example, the interface 32 is configured to display a first module 40 for multiple different patients 6 in multiple different rooms 2 connected to multiple different medical devices 4, whereby a second module 50 (and/or third, fourth, fifth etc. modules) may be selectively displayed for a given one of the patients.

As discussed above, the system incorporates a computing system CS100, and particularly a processing system CS110 (FIG. 7) that determines which of the plurality of supplemental datasets 54 to present on the interface 32 as the selected supplemental data 56 based on the alarm being triggered on the medical device 4.

By presenting the specific select supplemental data 56 chosen to correspond to the present alarms on the medical device 4, the one or more alarm limits corresponding to that alarm may be updated to manage the alarm of the medical device 4 more accurately to prevent false alarms. For example, if an alarm is active on a medical device indicating that the heart rate, as the select active data 46 has gone to 0, it may be helpful for the caregiver to automatically select an A/V feed 57A to provide among the select supplemental data 56 in the second module 50 on the same interface 32. This enables the caregiver to immediately see that the patient 6 has exited the bed 8 and disconnected all connections 10, thereby allowing the caregiver to quickly recognize that no cardiac incidences have occurred, but rather that the patient 6 has disconnected themselves from the medical device 4 altogether. In this example, the caregiver may not need to adjust alarm limits, but the automatic selection of appropriate select supplemental data 56 based on the alarm provides for quick resolution and management of the alarms state.

In another example, also referencing FIG. 2, a high heart rate alarm may be present within the first module 40, which through assessment through by the processing module CS110 may result in selection of information for medication administered 57B as select supplemental data 56 to display within the second module 50. In this case, the caregiver is prompted to review the recent medications provided to the patient to see if any such medications would be expected to have an impact on the heart rate as provided in the first module 40, and thus is the cause of the alarm. In certain examples, the caregiver may then adjust the alarm limits for the heart rate alarm in recognition of the medication administered to the patient, thereby preventing false alarms while this medication is active in the patient's system. In certain examples, the system 30 provides for automatically providing suggested adjustments to the alarm limits based on the information provided and the medication administered 57B.

FIG. 2 further shows a third module 60 on the interface 32, which is configured to present historic data 62. The historic data 62 presented is specifically select historic data 66 chosen from among a plurality of historic data sets 64 based on the alarm present on the medical device 4. In certain examples, the system 30 provides for choosing the select historic data 66 to present in third module 60 based one or more alarms in present, in the example of FIG. 2 showing historic data for the heart rate, temperature, and respiratory rate of the patient over time. Clinical events 63 may be overlaid one or more of the select historic data 66 being presented, such as the administration of medication, getting the patient out of bed or performing a procedure, and/or the like. In the example shown, the upper limit 16 and lower limit 18 of the alarm limits for the alarm are also shown super imposed on the select historic data 66, which enables the caregiver to identify patterns as to when the select historic data 66 exceeds the alarm limits, including in conjunction with the clinic events 63. This allows the caregiver to adjust the alarm limits as necessary to once again reduce false alarms.

In certain examples, the system 30 automatically identifies patterns within the historic data 62, which feed into subsequent suggestions for the caregiver adjusting the alarm limits when the system 30 recognizes these patterns in subsequent times. This may involve deep learning and/or artificial intelligence based on training in a manner known in the art.

According to an aspect of the disclosure, a caregiver input indicating that an alarm is false may be compared to the selected historic data to identify patterns. The patterns identified may be stored as false alarm predictions accessible by the system. Once false alarm predictions are stored, the system may analyze the selected historic data using the false alarm predictions and, based on the analysis, present a suggestion to check whether the alarm is false when the patterns are identified in the selected historic data based on the false alarm predictions.

The exemplary system 30 of FIG. 2 further shows a fourth module 70 on the interface 32 that presents alarm frequency data 72. In the example shown, this alarm frequency data 72 may be shown as single-sided data 73B or double-sided data 73A corresponding to whether one or two alarm limits are present. In the example shown, the upper limit 16 and/or lower limit 18 are shown such that the caregiver may ascertain how far beyond the alarm limits the active data 42 triggering the alarm had been over time. In other words, the alarm frequency data 72 may show whether the active data 42 goes well beyond the alarm limits, or barely exceeds these limits, and what percentage of the alarm frequency falls at each distance from the upper and lower limits. This again helps the caregiver reduce false alarms by perhaps making small adjustments to the upper or lower limit that would greatly reduce the incidence of alarm without having negative effects on the care given to the patient. This allows a remote caregiver to communicate back to the bedside team, allowing for a continuum of patient care on any clinically significant patient care notes. The present inventors have further identified that in certain examples it is advantageous for the user to drag the upper limit 16 and/or lower limit 18 in the alarm frequency data 72. To see a hypothetical change in the alarm frequency if different limits were set for the medical device 4.

The interface 32 of FIG. 2 further shows a fifth module 80 presenting select actions 84 from among a plurality of possible actions 82 based on the active data 42, supplemental data 52, and/or historic data 62 discussed above. In the example shown, the select actions 84 include generating a prompt or automatically triggering a digital communication message 87A to the responsible clinician, which in certain examples includes the alarms being currently triggered, the alarm limits, and relevant supplemental data 52 and/or historic data 62 to provide context to the clinician to ascertain whether changes to the alarm limits (or clinical intervention) are necessary. Another example of select action 84 shown in FIG. 2 is writing to the EMR 87B which may be again provided as a prompt for the user or may occur automatically.

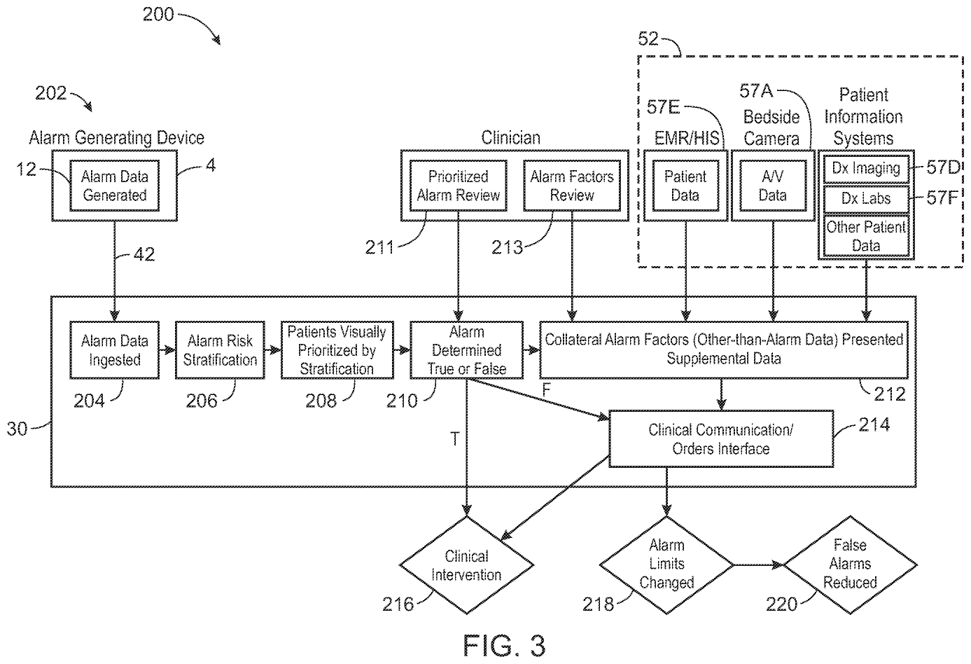

FIG. 3 depicts an exemplary process flow 200 for managing alarms according to an example. Operation 202 begins with an alarm being generated at the medical device 4, which is provided as active data 42 and ingested in operation 204 by the system 30. The system 30 provides for stratifying the alarm risk in operation 206, specifically applying logic to characterize the alarm as red (an immediate need for clinical intervention), yellow (a lower-urgency need for medical intervention), or green (no imminent clinical intervention needed). This stratification may be performed by reference to different rules stored within the memory system CS120 accessible by the processing system CS110 (FIG. 7) in a manner known in the art.

In examples in which the interface 32 provides data for multiple patients, operation 208 provides for visually prioritizing the patient's best ratification, for example showing the priority rating 94 as shown in the first module 40 or FIG. 2, based on the alarm's risk stratification of operation 206.

Operation 210 then provides for determining whether the alarm is true or false, which may be prompted to the user by a priority prompt 95 within the first module 40 as shown in FIG. 2, performed the clinician as operation 211. If operation 210 indicates that the alarm is true, clinical interventions provided in operation 216 in a manner presently known in the art. If instead the clinician indicates that the alarm is false in operation 210, the process continues to the operation 212, whereby the clinician reviews (operation 213) the supplemental data previously discussed to assess whether the alarm limits are set appropriately in view of the alarm being false. This determination includes consideration of a variety of supplemental data 52, including the examples of AV data 57A, EMR/HIS data 57E, imagining results 57D, lab results 57F, and other patient data as discussed above.

The result may be a clinical communication or changes to the orders interface in operation 214, particularly if the alarm limits are set based on orders. In other words, in certain examples the alarms are set based on standing protocols or orders from a caregiver and thus require a revised order before modifying. The alarm limits are then changed in operation 218 in recognition of the analysis of the supplemental data in operation 212 and updates to the orders interface in operation 214, resulting in reduced false alarms in operation 220 to thereby reduce alarm fatigue.

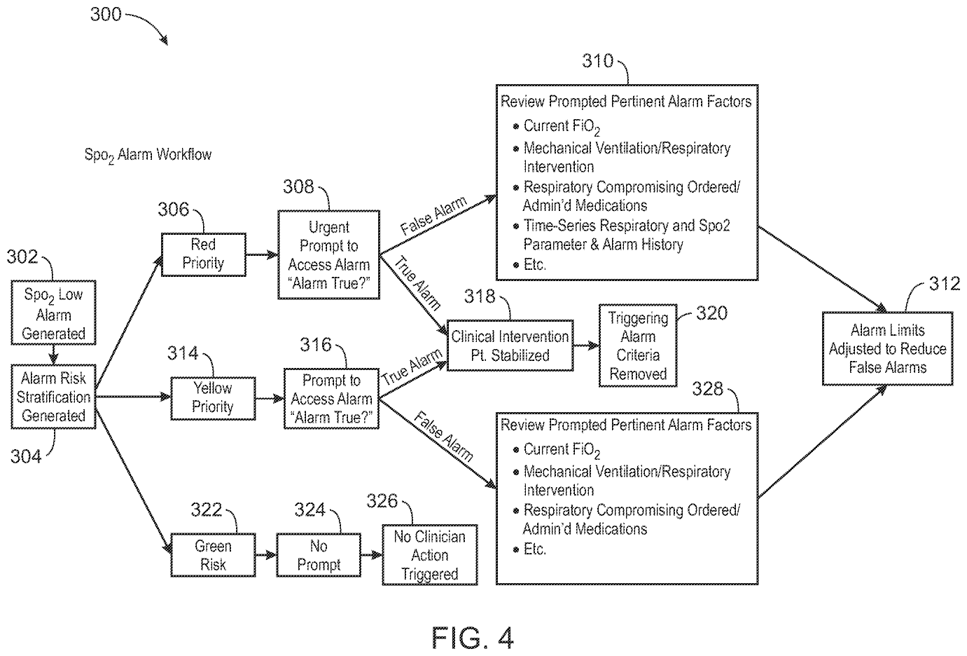

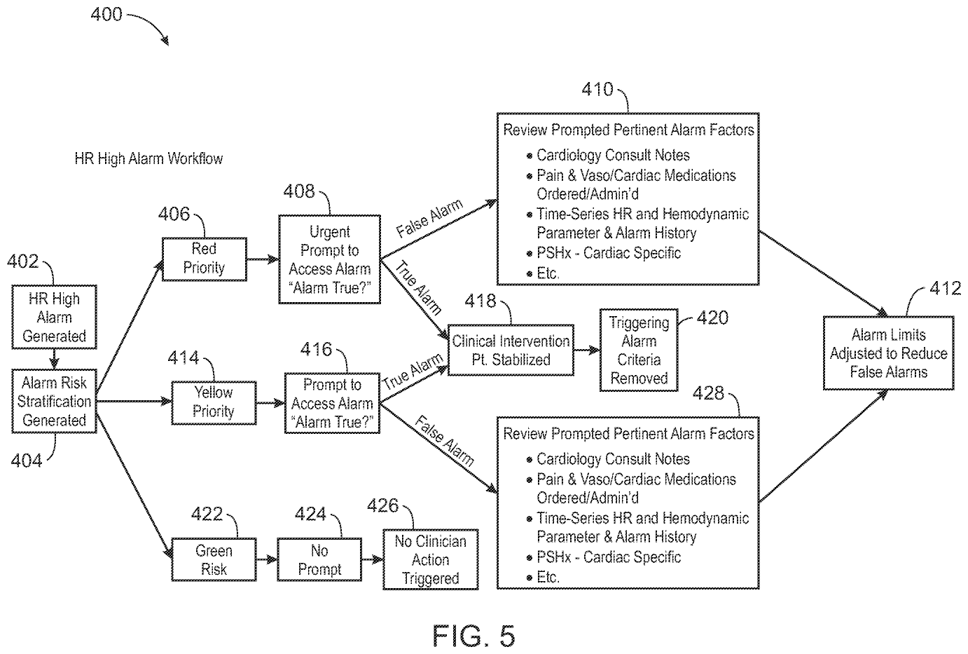

FIGS. 4 and 5 present two additional examples of processes 300, 400 similar to that shown in FIG. 3 but for specific cases of an SP02 alarm workflow and a heart rate high alarm workflow, respectively. It will be recognized that like-numbers are used for like-operations unless otherwise provided between FIGS. 4 and 5. The alarms are generated in operations 302 and 402, which risk stratification generated in operations 304 and 404. If the risks are determined to be green, no prompt is necessary on the interface 32 and no clinician action triggered. If instead the risk is determined to be a yellow or intermediate priority in operations 314 and 414, the clinician is prompted in operations 316 and 416 to provide a user alarm input as to whether the alarms are true or false. If false, operations 328 and 428 provide for presenting supplemental data 52 in second module 50 (FIG. 2) to prompt the clinician to reassess whether the alarm limits should be changed to prevent further false alarms, which are adjusted in operations 312 and 412 accordingly. If instead the alarms are true, clinical intervention is provided in operations 318 and 418, thereby removing the triggering alarm criteria in operations 320 and 420.

If instead the risks of the alarm are determined to be red priority in operations 306 and 406, the caregiver receives an urgent prompt to assess whether the alarms are true in operations 308 and 408. If the alarms are true, the processes 300 and 400 proceed to operation 318 and 418 as previously discussed. If instead the alarms are indicated to be false, the processes continue to operations 310 and 410 whereby select supplemental data 56 provided in second module 50 in a manner previously discussed with respect to operations 328 and 428, thereby assisting the caregiver in adjusting the alarm limits in operations 312 and 412 as necessary.

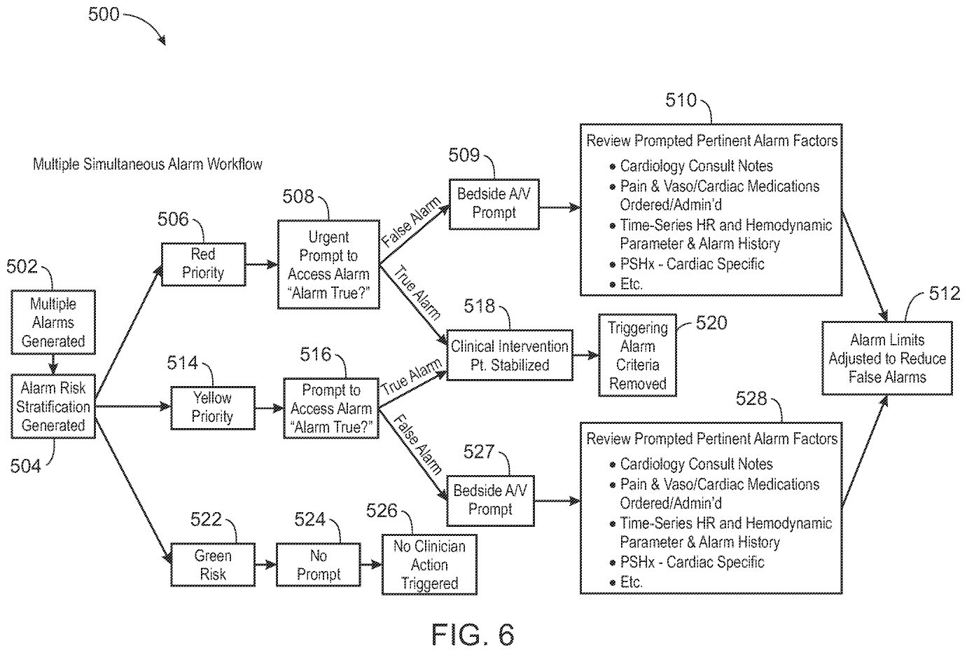

FIG. 6 provides for a process flow 500 similar to that previously discussed with FIGS. 4 and 5, but now having multiple alarms generated in operation 502. While the process flow 500 is generally similar to that previously discussed for FIGS. 4 and 5, additional distinctions are expressly discussed here. For example, the process flow 500 shows operations 509 and 527, which provide the exemplary supplemental data 52 of prompting the user to view the A/V feed 57A from the camera 20 in the room 2 of the patient 6. This may be particularly advantageous if the multiple alarms are determined by the processing systems CS110 to be contradictory, or in other words alarms which do not make sense to be going off simultaneously for example, if a first alarm indicates that the etCO2 is zero (appearing that the patient is not breathing since the end title CO2 is zero), and also an alarm or active data 42 indicating that the SpO2 is in the upper 90 s (indicating good blood profusion for the patient exists), is useful to view the patient via the A/V feed 57A to see if the patient 6 has removed one of the connections 10, since both states indicated by the alarms cannot simultaneously be true. Other supplemental data 52 may also be chosen according to the alarms being presented, including cardiology consultation notes, pain and/or vaso/cardiac medications have been ordered and/or administered or other information that should be reviewed by the caregiver above and beyond the active data 42 and ascertaining whether the limits of the alarms are appropriately set.

Certain aspects of the present disclosure are described or depicted as functional and/or logical block components or processing operations, which may be performed by any number of hardware, software, and/or firmware components configured to perform the specified functions. For example, certain embodiments employ integrated circuit components, such as memory elements, digital signal processing elements, logic elements, look-up tables, or the like, configured to carry out a variety of functions under the control of one or more processors or other control devices. The connections between functional and logical block components are merely exemplary, which may be direct or indirect, and may follow alternate pathways.

As shown in FIG. 7, in certain examples the control system CS100 communicates with each of the one or more components of the system 30 via a communication link CL, which can be any wired or wireless link. The control module CS100 is capable of receiving information and/or controlling one or more operational characteristics of the system 30 and its various sub-systems by sending and receiving control signals via the communication links CL. In one example, the communication link CL is a controller area network (CAN) bus; however, other types of links could be used. It will be recognized that the extent of connections and the communication links CL may in fact be one or more shared connections, or links, among some or all of the components in the system 30. Moreover, the communication link CL lines are meant only to demonstrate that the various control elements are capable of communicating with one another, and do not represent actual wiring connections between the various elements, nor do they represent the only paths of communication between the elements. Additionally, the system 30 may incorporate various types of communication devices and systems, and thus the illustrated communication links CL may in fact represent various different types of wireless and/or wired data communication systems.

The control system CS100 may be a computing system that includes a processing system CS110 with may include one or more processors, memory system CS120, and input/output (I/O) system CS130 for communicating with other devices, such as input devices CS99 and output devices CS101, either of which may also or alternatively be stored in a cloud 1002. The processing system CS110 loads and executes an executable program CS122 from the memory system CS120, accesses data CS124 stored within the memory system CS120, and directs the system 30 to operate as described in further detail below.

The processing system CS110 may be implemented as a single microprocessor or other circuitry, or be distributed across multiple processing devices or sub-systems that cooperate to execute the executable program CS122 from the memory system CS120. Non-limiting examples of the processing system include general purpose central processing units, application specific processors, and logic devices.

The memory system CS120 may comprise any storage media readable by the processing system CS110 and capable of storing the executable program CS122 and/or data CS124. This data CS124 may include various datasets described above, including groups of parameters by which individual datasets may be selected for display, tables of which supplemental data to display based on the alarms active for a patient, and/or algorithms for suggested alarm limit revisions based on analysis of the various sources of information, for example. The memory system CS120 may be implemented as a single storage device, or be distributed across multiple storage devices or sub-systems that cooperate to store computer readable instructions, data structures, program modules, or other data. The memory system CS120 may include volatile and/or non-volatile systems, and may include removable and/or non-removable media implemented in any method or technology for storage of information. The storage media may include non-transitory and/or transitory storage media, including random access memory, read only memory, magnetic discs, optical discs, flash memory, virtual memory, and non-virtual memory, magnetic storage devices, or any other medium which can be used to store information and be accessed by an instruction execution system, for example.

The functional block diagrams, operational sequences, and flow diagrams provided in the Figures are representative of exemplary architectures, environments, and methodologies for performing novel aspects of the disclosure. While, for purposes of simplicity of explanation, the methodologies included herein may be in the form of a functional diagram, operational sequence, or flow diagram, and may be described as a series of acts, it is to be understood and appreciated that the methodologies are not limited by the order of acts, as some acts may, in accordance therewith, occur in a different order and/or concurrently with other acts from that shown and described herein. For example, those skilled in the art will understand and appreciate that a methodology can alternatively be represented as a series of interrelated states or events, such as in a state diagram. Moreover, not all acts illustrated in a methodology may be required for a novel implementation. The terms “include,” “includes,” “have,” “having,” “comprise,” and “comprises” are intended to be open ended and therefore not preclude the addition of other elements.

This written description uses examples to disclose the invention, including the best mode, and also to enable any person skilled in the art to make and use the invention. Certain terms have been used for brevity, clarity, and understanding. No unnecessary limitations are to be inferred therefrom beyond the requirement of the prior art because such terms are used for descriptive purposes only and are intended to be broadly construed. The patentable scope of the invention is defined by the claims and may include other examples that occur to those skilled in the art. Such other examples are intended to be within the scope of the claims if they have features or structural elements that do not differ from the literal language of the claims, or if they include equivalent features or structural elements with insubstantial differences from the literal languages of the claims.

What is claimed is:

1. A system for managing an alarm based on active data from a medical device and based on supplemental data, wherein the active data has one or more alarm limits for triggering the alarm, the system comprising:
a display having a first window and a second window, wherein the first window is configured to present select active data from among a plurality of active datasets comprising the active data, the second window is configured to present select supplemental data from among a plurality of supplemental datasets comprising the supplemental data, wherein the alarm is triggered based on the active data;
a processing system configured to:
determine which of the plurality of supplemental datasets to present as the select supplemental data in the second window based on the alarm triggered;
control the display to display the select supplemental datasets in the second window; and
based on receiving a user alarm input indicating that the alarm is false, provide a prompt for updating the one or more alarm limits based on the displayed select supplemental data to manage the alarm of the medical device.

2. The system according to claim 1, wherein the select active data includes a heart rate, and wherein the plurality of supplemental data includes at least one of an A/V feed of a room, laboratory results, medication ordered, and medication administered.

3. The system according to claim 1, wherein the plurality of supplemental data includes at least one of medication ordered and medications administered,
the system further comprising a medication information database accessible by the processing system, wherein the processing system analyzes the alarm in view of the at least one of the medication ordered and the medication administered and the medication information database to determine whether to present the at least one of the medication ordered and the medication administered among the select supplemental data in the second window.

4. The system according to claim 1, wherein the processing system is further configured to, based on receiving a user alarm input indicating that the alarm is false, change which of the plurality of supplemental datasets to present among the select supplemental data in the second window based on the alarm being indicated to be false.

5. The system according to claim 4, wherein the processing system is further configured to provide a prompt requesting the user alarm input based on the alarm being present on the medical device.

6. The system according to claim 1, wherein the processing system is further configured to determine, based on a trained neural network model, which of the plurality of active datasets to be present as the select active data based on the alarm of the medical device, the neural network model being trained based on a correlation between false positive alarms and supplemental datasets.

7. The system according to claim 1, wherein the medical device is among a plurality of medical devices corresponding to a plurality of patients, wherein the active data and the supplemental data for each of the plurality of patients are receivable and presentable on the display, and wherein the processing system is further configured to assign and present a priority rating to each of the plurality of patients based on the alarm present on the medical device corresponding thereto.

8. The system according to claim 7, wherein the processing system is further configured to receive a patient selection selecting among the plurality of patients, and wherein the select supplemental data displayed on the display corresponds to which of the plurality of patients is selected.

9. The system according to claim 1, the processing system is further configured to control the display to display present selected historic data in a third window,
wherein the selected historic data is among a plurality of historic datasets received from the medical device over time, and
wherein the processing system is further configured to:
determine which of the plurality of historic datasets to present as the selected historic data based on the alarm present for the medical device,
control the display to display the selected historic data in the third window; and
based on displaying the selected historic data, provide the prompt for updating the one or more alarm limits based on the selected historic data to manage the alarm of the medical device.

10. The system according to claim 9, wherein the processing system determines which of the plurality of supplemental datasets to present as the select supplemental data based also on the selected historic data presented.

11. The system according to claim 10, wherein the processing system is further configured to, based on receiving the user alarm input indicating that the alarm is false:
identify patterns in the historic data by comparing the indication that the alarm is false to the selected historic data, and
store the identified patterns as false alarm predictions that are accessible by the processing system.

12. The system according to claim 11, wherein the processing system is further configured to:
analyze the historic data using the false alarm predictions; and
based on the patterns being identified in the historic data, present a suggestion to check whether the alarm is false.

13. The system according to claim 10, wherein the processing system is further configured to identify patterns within the selected historic data and to correspondingly present medication administered among the select supplemental data in the second window.

14. The system according to claim 9, wherein the processing system is further configured to:
control the display to display alarm frequency data in a fourth window; and
determine the alarm frequency data based on how often the alarm is present within the selected historic data presented in the third window.

15. The system according to claim 14, wherein the alarm frequency data is further presented as a function of how much the selected historic data was outside the one or more alarm limits corresponding to the alarm, and
wherein the one or more alarm limits include an upper limit and a lower limit, and wherein the alarm frequency data is further presented as a function of how often the selected historic data was above the upper limit and below the lower limit.

16. The system according to claim 15, wherein the alarm frequency data further presents the upper limit and the lower limit, wherein the processing system is further configured to receive proposed change inputs for the upper limit and the lower limit, and wherein the alarm frequency data is updateable based on the user alarm inputs received.

17. The system according to claim 1, wherein a plurality of possible actions are accessible by the processing system, and wherein the processing system is further configured to control the display to display the select actions in a fifth window, wherein the select actions are among the plurality of possible actions and are selected by the processing system based on the alarm, the select actions comprising sending a communication to a caregiver presenting the alarm and requesting confirmation of the one or more alarm limits corresponding thereto.

18. The system according to claim 18, wherein the processing system is further configured to control the display to display selected historic data in a third window, wherein the selected historic data is among a plurality of historic datasets received from the medical device, wherein the processing system determines which of the plurality of historic datasets to present among the selected historic data based on the alarm on the medical device, and wherein the select actions include presenting suggested adjustments to the one or more alarm limits based on the selected historic data.

19. A patient monitoring device, comprising:
a communication interface configured to receive and send data;
a memory storing one or more instructions;
a display for displaying information to a user;
a user interface configured to receive inputs from the user; and
a processor configured to execute the one or more instructions to:
receive active data from one or more medical devices through the communication interface;
trigger an alarm based on the active data exceeding a threshold;
based on the alarm being triggered, select, using a selection model, one or more types of supplemental data, from among a plurality of supplemental data types based on a type of the triggered alarm; and
automatically control the user interface to display the selected one or more types of supplemental data, the selected one or more types of supplemental data being displayed in conjunction with the active data.

20. A method of monitoring medical devices comprising:
receiving active data from a plurality of medical devices;
displaying the active data on a display;
triggering an alarm based on the active data from a medical device among the plurality of medical devices reaching one or more alarm limits;
based on the alarm being triggered, providing an interface for selecting whether the alarm is true of false; and
based on receiving a user alarm input indicating that the alarm is false:
selecting, using a selection model, one or more types of supplemental data, from among a plurality of supplemental data types based on a type of the triggered alarm; and
providing an interface for updating the one or more alarm limits to manage the alarm of the medical device.
